(19) United States
(12) Patent Application Publication
(10) Pub. No.: US 2025/0118443 A1
(43) Pub. Date: Apr. 10, 2025

(54) SYSTEMS AND METHODS FOR DETERMINING KIDNEY CONDITION BASED ON ELECTRICAL IMPEDANCE TOMOGRAPHY

(71) Applicant: Gense Technologies Limited, Wong Chuk Hang (HK)

(72) Inventors: Chung San Wong, Tseung Kwan O (HK); Russell Wade Chan, Kowloon (HK); Fedi Zouari, Tseung Kwan O (HK); Dipyaman Modak, Mong Kok (HK); Pak Heng Justin Chan, Pok Fu Lam (HK); Pak To Cheung, North Point (HK)

(21) Appl. No.: 18/832,299

(22) PCT Filed: Jan. 28, 2023
(86) PCT No.: PCT/CN2023/073584

§ 371 Date: Jul. 23, 2024

(30) Foreign Application Priority Data
Jan. 25, 2022 (HK) 32022047017.5

(51) Int. Cl. G16H 50/30; A61B 5/00; A61B 5/0536; A61B 5/20; G16H 10/60; G16H 50/20

(52) U.S. Cl. CPC G16H 50/30; A61B 5/0536; A61B 5/201; A61B 5/4842; G16H 10/60; G16H 50/20

(57) ABSTRACT

A computer-implemented method that includes processing a EIT data set of a subject to determine one or more kidney-related conductivity characteristics of the subject, and, determining, based on at least the one or more determined kidney-related conductivity characteristics of the subject, a health state or condition of the at least one kidney of the subject.

### TECHNICAL FIELD

The invention relates to determining kidney condition(s) based on electrical impedance tomography (EIT).

### BACKGROUND

Electrical impedance tomography is a medical imaging technique that can be used for determining electrical conductivity, permittivity, and/or impedance of a body part of a subject (animal, human, etc.).

### SUMMARY OF THE INVENTION

In a first aspect, there is provided a computer-implemented method that includes processing a EIT data set of a subject to determine one or more kidney-related conductivity characteristics of the subject, and determining, based on at least the one or more determined kidney-related conductivity characteristics, a health state or condition of the at least one kidney of the subject.

Optionally, the determining comprises: determining, based on at least the one or more determined kidney-related conductivity characteristics, whether the subject has a kidney disease. Optionally, the determining further comprises classifying a stage or a severity of the kidney disease.

Optionally, the determining comprises: determining, based on at least the one or more determined kidney-related conductivity characteristics, a value associated with an estimated glomerular filtration rate (GFR) of the subject. The value associated with an estimated glomerular filtration rate of the subject may be the estimated glomerular filtration rate of the subject or a value arithmetically-related to the estimated glomerular filtration rate of the subject.

Optionally, the determining comprises: processing, at least, the one or more determined kidney-related conductivity characteristics of the subject and one or more anthropometric characteristics of the subject, using a machine learning based processing model, to determine a quantitative or qualitative parameter associated with the health state or condition of the at least one kidney of the subject.

Optionally, the determining comprises: processing, using a machine learning based processing model, (i) the one or more determined kidney-related conductivity characteristics of the subject, (ii) one or more anthropometric characteristics of the subject, and (iii) one or more determined kidney-related conductivity characteristics of one or more reference subjects and/or one or more determined kidney-related conductivity characteristics of a group containing the subject and the one or more reference subjects, to determine a quantitative or qualitative parameter associated with the health state or condition of the at least one kidney of the subject. Preferably, the one or more reference subjects do not suffer from any kidney disease.

Optionally, the machine learning based processing model comprises a regression model.

Optionally, the regression model comprises a linear regression model, such as a Lasso model, etc.

Optionally, the regression model comprises a non-linear regression model, such as decision trees, random forest, etc.

Optionally, the machine learning based processing model comprises a classification model.

Optionally, the one or more anthropometric characteristics comprise, or are related to, one or more of: age of the subject, weight of the subject, height of the subject, and waist circumference of the subject. The one or more anthropometric characteristics may comprise, or be related to, one or more other anthropometric characteristics.

Optionally, the quantitative or qualitative parameter associated with the health state or condition of the at least one kidney of the subject comprises: a value associated with an estimated glomerular filtration rate of the subject, e.g., an estimated glomerular filtration rate score of the subject and an arithmetically-related score/value.

Optionally, the determining further comprises: comparing the quantitative or qualitative parameter associated with the health state or condition of the at least one kidney of the subject with reference parameter data (reference parameter value(s) or range(s)) to determine whether the subject has a kidney disease.

Optionally, the determining further comprises: classifying, based on the comparing, a stage or a severity of the kidney disease.

Optionally, the kidney disease is a chronic kidney disease.

Optionally, the EIT data set contains EIT data obtain from an abdominal region of the subject.

Optionally, the EIT data set is obtained by (a) providing excitation signals (voltage, potential, current, etc.) at a frequency to the subject via electrodes attached to the abdominal region of the subject, (b) measuring responsive signals (voltage, potential, current, etc.) received via the electrodes as a result of the providing of the excitation signals, and (c) repeating steps (a) and (b) for a plurality of frequencies. The EIT data set may comprise a plurality of EIT data subsets each associated with a respective one of the plurality of frequencies.

Optionally, the processing comprises: processing the EIT data set to obtain a processed EIT data set (which includes a plurality of processed EIT data subsets), and processing the processed EIT data set to obtain a frequency difference EIT data set (which includes a plurality of frequency difference EIT data subsets). The plurality of frequency difference EIT data subsets may correspond to a plurality of conductivity maps.

Optionally, the processing comprises: performing a group source separation operation using the frequency difference EIT data set and one or more reference frequency difference EIT data sets of corresponding one or more reference subjects to determine kidney-related component of the frequency difference EIT data set and kidney-related component of each of the one or more reference frequency difference EIT data sets.

Optionally, the processing further comprises: performing a conductivity characteristics extraction operation using the kidney-related component of the frequency difference EIT data set and optionally the kidney-related component of each of the one or more reference frequency difference EIT data sets to determine at least the one or more kidney-related conductivity characteristics of the subject.

Optionally, the processing of the EIT data set comprises: filtering and/or smoothing each of the plurality of EIT data subsets.

Optionally, the processing of the EIT data set comprises: processing the EIT data set using a classifier model (e.g., a machine learning based processing model) to determine respective performance of each of the plurality of electrodes, the performance being associated with quality of responsive signals or data obtained from the respective electrode, and preventing the responsive signals or data obtained via any one or more of the plurality of electrodes determined to have insufficient performance from being included in the processed EIT data set. In some examples, a respective performance score is determined for each of the plurality of electrodes and the respective performance scores are compared with a reference data to determine whether the any of (and if so which) of the electrodes provided insufficient performance when the data is taken. In some examples, the classification model can determine the electrodes that have insufficient performance when the data is taken.

Optionally, the processing of the processed EIT data set comprises: determining, for each respective one or more of the plurality of processed EIT data subsets, respective difference between the respective processed EIT data subset and a reference EIT data subset, so as to obtain the plurality of frequency difference EIT data subsets each associated with a respective one of a difference between the respective processed EIT data subset and a reference EIT data subset.

Optionally, the reference EIT data subset comprises at least one of the plurality of processed EIT data subsets. In some examples, at least one of the plurality of processed EIT data subsets is respectively used as the reference EIT data subset.

Optionally, the processing comprises: processing the EIT data set to obtain a frequency difference EIT data set (which includes a plurality of frequency difference EIT data subsets). The plurality of frequency difference EIT data subsets may correspond to a plurality of conductivity maps. Optionally, the processing further comprises: performing a group source separation operation using the frequency difference EIT data set and one or more reference frequency difference EIT data sets of corresponding one or more reference subjects to determine kidney-related component of the frequency difference EIT data set and kidney-related component of each of the one or more reference frequency difference EIT data sets. Optionally, the processing further comprises: performing a conductivity characteristics extraction operation using the kidney-related component of the frequency difference EIT data set and optionally the kidney-related component of each of the one or more reference frequency difference EIT data sets to determine at least the one or more kidney-related conductivity characteristics of the subject.

Optionally, the performing of the group source separation operation comprises: performing a dimensionality reduction operation on the frequency difference EIT data set and one or more reference frequency difference EIT data sets of corresponding one or more reference subjects to determine kidney-related component of the frequency difference EIT data set and respective kidney-related component of each of the one or more reference frequency difference EIT data sets.

Optionally, the performing of the conductivity characteristics extraction operation comprises: determining, using the kidney-related component of the frequency difference EIT data set, the one or more kidney-related conductivity characteristics of the subject.

Optionally, the one or more kidney-related conductivity characteristics of the subject comprises one or more statistical kidney-related conductivity characteristics of the subject.

Optionally, the one or more statistical kidney-related conductivity characteristics of the subject comprises at least one of: an average of conductivity characteristics in a defined kidney region within an area; an average of conductivity characteristics outside the defined kidney region within the area; and an average of conductivity characteristics within the area. The area may be an area within the conductivity map.

Optionally, the performing of the conductivity characteristics extraction operation comprises: determining, using the kidney-related component of the frequency difference EIT data set and respective kidney-related component of each of the one or more reference frequency difference EIT data sets, one or more kidney-related conductivity characteristics of a group containing the subject and the one or more reference subjects. And, the determining of the health state or condition of the at least one kidney of the subject may be further based on the one or more kidney-related conductivity characteristics of the group.

Optionally, the one or more kidney-related conductivity characteristics of the subject comprises one or more statistical kidney-related conductivity characteristics of the group.

Optionally, the one or more statistical kidney-related conductivity characteristics of the group comprises at least one of: an average of conductivity characteristics in a defined kidney region within an area, an average of conductivity characteristics outside the defined kidney region within the area, and an average of conductivity characteristics both in and outside the defined kidney region within the area. The area may be an area within the conductivity map (e.g., averaged conductivity map determined from the conductivity maps).

In a second aspect, there is provided a system comprising one or more processors and memory storing one or more programs configured to be executed by the one or more processors. The one or more programs include instructions for performing or facilitating performing of the computer-implemented method of the first aspect.

In a third aspect, there is provided a non-transitory computer-readable storage medium storing one or more programs configured to be executed by one or more processors. The one or more programs include instructions for performing or facilitating performing of the computer-implemented method of the first aspect.

In a fourth aspect, there is provided a computer program product comprising instructions which, when the computer program is executed by a computer, cause or facilitate the computer to carry out the computer-implemented method of the first aspect.

In a fifth aspect, there is provided a computer-implemented method, comprising: processing electrical impedance tomography data (including kidney data) obtained from a subject to determine conductivity characteristic associated with a kidney of the subject; and determining, based on the determined conductivity characteristic, a health state or condition of the kidney of the subject.

Optionally, the determining comprises determining, based on the determined conductivity characteristic, whether the subject is suffering from kidney disease (e.g., chronic kidney disease).

Optionally, the determining further comprises classifying a stage of the kidney disease (e.g., chronic kidney disease).

Optionally, the determining comprises determining, based on the determined conductivity characteristic, a glomerular filtration rate score or an estimated glomerular filtration rate score of the subject.

Optionally, the determining comprises comparing the determined conductivity characteristic with predetermined mapping table/curve/graph/relationship between different conductivity characteristics and their respective glomerular filtration rate score or an estimated glomerular filtration rate score.

Optionally, the determining comprises determining, based on the determined conductivity characteristic, a glomerular filtration rate or an estimated glomerular filtration rate of the subject.

Optionally, the determining comprises comparing the determined conductivity characteristic with predetermined mapping table/curve/graph/relationship between different conductivity characteristics and their respective glomerular filtration rate or an estimated glomerular filtration rate.

Optionally, the processing further comprises: filtering and/or denoising the electrical impedance tomography data.

Optionally, the processing further comprises: reconstructing EIT images associated with the kidney of the subject, each of the EIT image being associated with a respective frequency of the excitation signal; and determining respective conductivity maps based on the EIT images.

Optionally, the electrical impedance tomography data (including kidney data) are electric potential data obtained from electrodes attached to the subject (e.g., the upper abdominal region of the subject).

Optionally, the electrical impedance tomography data (including kidney data) comprises multiple sets of electric potential data each obtained for an excitation signal of a respective frequency, and wherein the frequency for the different sets are different.

Optionally, one of the set is a reference set, and the processing further comprises determining respective differences between the reference set and each of the other sets, to obtain multiple sets of electric potential difference data.

Optionally, the processing further comprises determining the conductivity characteristic (e.g., conductivity changes) based on the multiple sets of electric potential difference data.

Optionally, the subject is human.

In a sixth aspect, there is provided a system, comprising one or more processors arranged (e.g., programmed) to: process electrical impedance tomography data (including kidney data) obtained from a subject to determine conductivity characteristic associated with a kidney of the subject; and determine, based on the determined conductivity characteristic, a health state or condition of the kidney of the subject.

The system may further include an output device (e.g., a display) for presenting the determination result to the user.

Optionally, the one or more processors are arranged to determine, based on the determined conductivity characteristic, whether the subject is suffering from kidney disease (e.g., chronic kidney disease).

Optionally, the one or more processors are arranged to classify a stage of the kidney disease (e.g., chronic kidney disease).

Optionally, the one or more processors are arranged to determine, based on the determined conductivity characteristic, a glomerular filtration rate score or an estimated glomerular filtration rate score of the subject.

Optionally, the one or more processors are arranged to compare the determined conductivity characteristic with predetermined mapping table/curve/graph/relationship between different conductivity characteristics and their respective glomerular filtration rate score or an estimated glomerular filtration rate score.

Optionally, the one or more processors are arranged to determine, based on the determined conductivity characteristic, a glomerular filtration rate or an estimated glomerular filtration rate of the subject.

Optionally, the one or more processors are arranged to compare the determined conductivity characteristic with predetermined mapping table/curve/graph/relationship between different conductivity characteristics and their respective glomerular filtration rate or an estimated glomerular filtration rate.

Optionally, the one or more processors are arranged to filter and/or denoise the electrical impedance tomography data.

Optionally, the one or more processors are arranged to reconstruct EIT images associated with the kidney of the subject, each of the EIT image being associated with a respective frequency of the excitation signal; and determine respective conductivity maps based on the EIT images.

Optionally, the electrical impedance tomography data (including kidney data) are electric potential data obtained from electrodes attached to the subject (e.g., the upper abdominal region of the subject).

Optionally, the electrical impedance tomography data (including kidney data) comprises multiple sets of electric potential data each obtained for an excitation signal of a respective frequency, and wherein the frequency for the different sets are different.

Optionally, one of the set is a reference set, and the processing further comprises determining respective differences between the reference set and each of the other sets, to obtain multiple sets of electric potential difference data.

Optionally, the one or more processors are arranged to determine the conductivity characteristic (e.g., conductivity changes) based on the multiple sets of electric potential difference data.

Optionally, the subject is a human.

In a seventh aspect, there is provided a non-transitory computer-readable medium storing computer instructions that, when executed by one or more processors, causes the one or more processors to perform the method of the first aspect.

In an eighth aspect, there is provided a computer program product comprising instructions which, when the program is executed by a computer, cause the computer to carry out the method of the first aspect.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

Terms of degree such that “generally”, “about”, “substantially”, or the like, are used, depending on context, to account for manufacture tolerance, degradation, trend, tendency, imperfect practical condition(s), etc. For example, when a value is modified by terms of degree, such as “about”, such expression may include the stated value ±10%, ±5%, ±2%, or ±1%.

Unless otherwise specified, the terms “connected”, “coupled”, “mounted”, and the like, are intended to encompass both direct and indirect connection, coupling, mounting, etc. Unless other specified, or context required otherwise, the term “conductivity”, and the like, means electrical conductivity or bio-conductivity.

### BRIEF DESCRIPTION OF THE DRAWINGS

Embodiments of the invention will now be described, by way of example, with reference to the accompanying drawings in which:

FIG. 1 is a flowchart illustrating a computer-implemented EIT data processing method in some embodiments of the invention;

FIG. 2 is a flowchart illustrating a computer-implemented EIT data processing method in some embodiments of the invention;

FIG. 3 is a schematic diagram illustrating a computer-implemented EIT data processing method in some embodiments of the invention;

FIG. 4 is a schematic diagram illustrating an example operation including EIT data acquisition, processing, and analysis in one embodiment of the invention;

FIG. 5A shows a picture of a water phantom with a pig kidney for use in an example experiment;

FIG. 5B illustrates a series of EIT images of the water phantom with the pig kidney with respect to a reference water phantom (without the pig kidney) obtained in the example experiment (the labelled stimulation frequencies are applied to obtain the EIT images);

FIG. 5C illustrates a series of frequency difference EIT (fd-EIT) images of the water phantom with a pig kidney relative to a reference water phantom (without the pig kidney) (stimulation frequency 10 kHz) obtained in the example experiment (the labelled stimulation frequencies are applied to obtain the EIT images before frequency differencing);

FIG. 6A are graphs showing a relationship between standard, calculated estimated glomerular filtration rate (eGFR) values and different stages of chronic kidney disease and a relationship between EIT-measured conductivity changes and different stages of chronic kidney disease in one example;

FIG. 6B is a graph (scatter plot) showing a relationship of standard, calculated eGFR values with EIT-measured conductivity changes in one example;

FIG. 6C are graphs showing a relationship between EIT-measured conductivity changes and different severities of chronic kidney disease and a relationship between EIT-determined eGFR score and different severities of chronic kidney disease in one example;

FIG. 6D is a graph (scatter plot) showing a relationship of standard, calculated eGFR values (true eGFR) with EIT-determined eGFR score in one example;

FIG. 7A is a schematic diagram illustrating an example operation including EIT data acquisition, processing, and analysis in one embodiment of the invention;

FIG. 7B is a schematic diagram illustrating an example EIT data processing in one embodiment of the invention;

FIG. 8A is a graph showing linear correlation coefficients and relative importance of various EIT-features (conductivity characteristics) and anthropometric features of the subject in one example;

FIG. 8B is a graph (scatter plot) showing a relationship and classification specificity and sensitivity of a eGFR regression model (associated with true (blood test) eGRF value and EIT-determined eGFR score) in one embodiment of the invention;

FIG. 8C is a graph showing a receiver operating characteristic (ROC) curve of a eGFR regression model and classification scheme in one embodiment of the invention;

FIG. 8D are graphs showing EIT-determined eGFR score at different stages (S1-S5) and severities of chronic kidney disease in one example;

FIG. 9A is a graph showing simulation results obtained based on existing population eGFR distribution and error of the model in one embodiment of the invention;

FIG. 9B is a graph (scatter plot) showing simulated relationship and classification specificity and sensitivity of a eGFR regression model (associated with true (blood test) eGRF value and simulated EIT-determined eGFR score) in one embodiment of the invention;

FIG. 9C are graphs showing simulated EIT-determined eGFR score at different stages (S1-S5) and severities of chronic kidney disease in one example;

FIG. 10 is a block diagram of a data processing system arranged to perform one or more of the method embodiments (partly or entirely) in some embodiments of the invention;

FIG. 11 is a block diagram of an example EIT system in some embodiments of the invention;

FIG. 12 is a block diagram of an example EIT console in some embodiments of the invention; and

FIG. 13 is a block diagram of an example EIT console in some embodiments of the invention.

### DETAILED DESCRIPTION

Inventors of this invention have realized, through their research, that kidney disease such as chronic kidney disease (CKD) is a common health problem in some territories. Generally, all renal abnormalities affecting kidney functions and structures, which last for more than three months, are considered as CKD. Problematically, CKD may progress and comorbid with other health related issues such as hypertension, diabetes, and cardiovascular disease. This makes CKD costly and/or difficult to treat. Inventors of this invention have devised, that early diagnosis/detection and treatment monitoring (e.g., longitudinal monitoring) of kidney disease such as CKD would be useful.

Inventors of this invention have realized, through their research, that conventional evaluation or monitoring of CKD relies on measurement of glomerular filtration rate (GFR) in urine sample or quantifying estimated GFR (eGFR) in blood serum samples, and that the current clinical standard method for classifying chronic kidney disease (CKD) is by evaluating the eGFR calculated by the standardized serum creatinine level from the collected blood samples. Generally, a lower eGFR indicates a more severe CKD stage. Inventors of this invention have realized, through their research, that this blood sample approach may be susceptible to bias and errors, e.g., in high eGFR range and/or may be susceptible to overestimation in early CKD stages, e.g., as biased by gender and muscle mass of the patient.

The inventors of the invention have devised, through their own research, trials, and experiments, that: kidney disease progression results in renal function deteriorations (e.g., reduced blood perfusion, restricted fluid diffusion, etc.), which would consequently lead to eGFR decrease and kidney tissue changes that causes fundamental dielectric parameters changes such as bio-conductivity. The conductivity of the kidney tissues may exhibit contrast in response to different frequencies of stimulations (e.g., alternating current (AC) stimulations).

Against this background the inventors of the invention have come up with the idea of using the contrast of electrical responses in one or both kidneys of the patient to evaluate the eGFR and/or to classify CKD stages. More generally, the inventors of the invention have come up with the idea of using electrical impedance tomography to assess the conductivity characteristics associated with the kidney(s), to determine a health state or condition of the kidney(s).

Electrical impedance tomography (EIT) is a non-invasive, ionizing radiation-free, and cost-effective imaging technique that can capture dielectric parameters within the body by sensing electrical signals from surface electrodes attached to the body. EIT can be used to reconstruct the interior of a body by measuring electrical signals at the body surface. Clinical application of EIT is based on the dependence of conductivity among tissue types and A.C. current frequencies. In some implementations of EIT, small alternative currents (AC) are sequentially applied via electrodes at single or multiple frequencies to excite an electric field within the body and corresponding dielectric parameters (i.e., impedance, conductivity, permittivity etc.) could represented as potential difference could be detected from the electrodes.

Conductivity of biological tissues (including kidneys) may according to tissue types and frequencies of applied AC current. In respect of kidneys, healthy kidneys are water- and electrolyte-rich organ with good electrical conductivity whereas unhealthy kidneys (e.g., ones with fibrosis, a characteristic histopathological change in CKD, presented as replacement of normal kidney tissue with matrices and fibrous substances) could lead to reduced electrical conductivity. These differences could be detected using EIT-based techniques such as frequency-difference-EIT (fd-EIT). In some EIT examples, the conductivity of a normal kidney and a kidney in a CKD patient may respond differently at each frequency due to existence of fibrous substances from kidney fibrosis. Thus, frequency-difference-EIT (fd-EIT) may be used to detect the changes across kidney disease progression. As illustrated in the following examples, eGFR and CKD stage are highly correlated, and fd-EIT could be used to estimate the eGFR and determine the CKD stage of the patient from the reconstructed conductivity distribution image. Problematically, however, in the region where the kidneys are in, other tissues with significant frequency responses such as the liver, the intestines, and muscles may exist.

The frequency responses from these tissues may superpose with or otherwise affect the kidney signals.

Based on the above, embodiments of the invention provide a method to process EIT data for use in determining health state or condition of one or more kidneys of a subject. Some embodiments relate to a data processing pipeline to extract the kidney signals from EIT data (e.g., in vivo data). Some embodiments relate to a machine learning based processing model, e.g., regression model, for estimating the eGFR of CKD patients using EIT features and one or more anthropometric characteristics of the subject. Some embodiments relate to using the regression result to classify the CKD stage and/or severity of the patient. Some embodiments provide a method to extract kidney signals from the EIT data.

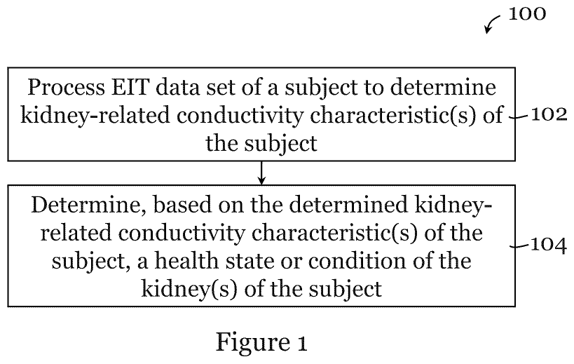

FIG. 1 shows a computer-implemented EIT data processing method 100 in some embodiments of the invention. The method includes, in step 102, processing a EIT data set of a subject to determine one or more kidney-related conductivity characteristics of the subject, and in step 104, determining a health state or condition of at least one kidney of the subject based at least on the one or more kidney-related conductivity characteristics of the subject. In some embodiments, steps 102 and 104 are performed separately (sequentially). In some embodiments, steps 102 and 104 are performed at least partly simultaneously.

In some embodiments, the EIT data set contains EIT data obtain from an abdominal region of the subject. In some embodiments, the EIT data set is obtained by (a) providing excitation signals (voltage, potential, current, etc.) at a frequency to the subject via electrodes attached to the abdominal region of the subject, (b) measuring responsive signals (voltage, potential, current, etc.) received via the electrodes as a result of the providing of the excitation signals, and (c) repeating steps (a) and (b) for different frequencies. The EIT data set may comprise multiple EIT data subsets each associated with a respective one of the frequencies.

In some embodiments, step 102 may include one or more or all of:
(i) processing the EIT data set to obtain a processed EIT data set (which includes multiple processed EIT data subsets)
(ii) processing the processed EIT data set to obtain a frequency difference EIT data set (which includes multiple frequency difference EIT data subsets, the frequency difference EIT data subsets may correspond to multiple conductivity maps.
(iii) performing a group source separation operation using the frequency difference EIT data set and one or more reference frequency difference EIT data sets of corresponding one or more reference subjects to determine kidney-related component of the frequency difference EIT data set and kidney-related component of each of the one or more reference frequency difference EIT data sets
(iv) performing a conductivity characteristics extraction operation using the kidney-related component of the frequency difference EIT data set and optionally the kidney-related component of each of the one or more reference frequency difference EIT data sets to determine at least the one or more kidney-related conductivity characteristics of the subject.

In some embodiments, (i) in step 102 may include filtering and/or smoothing each of the EIT data subsets.

In some embodiments, (i) in step 102 may additionally or alternatively include processing the EIT data set using a classifier model (e.g., a machine learning based processing model) to determine respective performance of each of the electrodes (the performance is associated with quality of responsive signals or data obtained from the respective electrode) when the EIT data is obtained and preventing the responsive signals or data obtained via any one or more of the electrodes determined to have insufficient performance from being included in the processed EIT data set. In some examples, a respective performance score is determined for each of the electrodes and the respective performance scores are compared with a reference data to determine whether the any of (and if so which) of the electrodes provided insufficient performance when the data is taken. In some examples, the classification model can determine the electrodes that have insufficient performance when the data is taken.

In some embodiments, (ii) in step 102 may include determining, for each respective one or more of the processed EIT data subsets, respective difference between the respective processed EIT data subset and a reference EIT data subset, so as to obtain the frequency difference EIT data subsets each associated with a respective one of a difference between the respective processed EIT data subset and a reference EIT data subset. At least one of the processed EIT data subsets may be respectively used as the reference EIT data subset.

In some embodiments, (iii) in step 102 may include performing a dimensionality reduction operation on the frequency difference EIT data set and one or more reference frequency difference EIT data sets of corresponding one or more reference subjects to determine kidney-related component of the frequency difference EIT data set and respective kidney-related component of each of the one or more reference frequency difference EIT data sets.

In some embodiments, (iv) in step 102 may include determining, using the kidney-related component of the frequency difference EIT data set, the one or more (e.g., statistical) kidney-related conductivity characteristics of the subject. For example, the one or more (e.g., statistical) kidney-related conductivity characteristics of the subject may include one or more of: an average of conductivity characteristics in a defined kidney region within an area, an average of conductivity characteristics outside the defined kidney region within the area, and an average of conductivity characteristics within the area. The area may be an area within the conductivity map. In some embodiments, other statistical kidney-related conductivity characteristics such as median, mode, standard deviation, etc., may be used instead of the mean.

In some embodiments, (iv) in step 102 may include determining, using the kidney-related component of the frequency difference EIT data set and respective kidney-related component of each of the one or more reference frequency difference EIT data sets, one or more (e.g., statistical) kidney-related conductivity characteristics of a group containing the subject and the one or more reference subjects. And, the determining of the health state or condition of the at least one kidney of the subject may be further based on the one or more kidney-related conductivity characteristics of the group. For example, the one or more (e.g., statistical) kidney-related conductivity characteristics of the group may include one or more of: an average of conductivity characteristics in a defined kidney region within an area, an average of conductivity characteristics outside the defined kidney region within the area, and an average of conductivity characteristics both in and outside the defined kidney region within the area. The area may be an area within the conductivity map (e.g., averaged conductivity map determined from the conductivity maps). In some embodiments, other statistical kidney-related conductivity characteristics such as median, mode, standard deviation, etc., may be used instead of the mean.

In some embodiments, step 104 includes: determining, based on at least the one or more determined kidney-related conductivity characteristics, whether the subject has a kidney disease. In some embodiments, step 104 also includes classifying a stage or a severity of the kidney disease.

In some embodiments, step 104 includes: determining, based on at least the one or more determined kidney-related conductivity characteristics, a value associated with an estimated glomerular filtration rate (GFR) of the subject. The value associated with an estimated glomerular filtration rate of the subject may be the estimated glomerular filtration rate of the subject or a value arithmetically-related to the estimated glomerular filtration rate of the subject.

In some embodiments, step 104 includes: processing, at least, the one or more determined kidney-related conductivity characteristics of the subject and one or more anthropometric characteristics of the subject, using a machine learning based processing model, to determine a quantitative or qualitative parameter associated with the health state or condition of the at least one kidney of the subject.

In some embodiments, step 104 includes: processing, using a machine learning based processing model, (i) the one or more determined kidney-related conductivity characteristics of the subject, (ii) one or more anthropometric characteristics of the subject, and (iii) one or more determined kidney-related conductivity characteristics of one or more reference subjects and/or one or more determined kidney-related conductivity characteristics of a group containing the subject and the one or more reference subjects, to determine a quantitative or qualitative parameter associated with the health state or condition of the at least one kidney of the subject. Preferably, the one or more reference subjects do not suffer from any kidney disease.

The machine learning based processing model may include a regression model, a classification model, etc. The regression model may include a linear regression model, such as a Lasso model, etc., or a non-linear regression model, such as decision trees, random forest, etc.

The one or more anthropometric characteristics may include or be related to one or more of, e.g., age of the subject, weight of the subject, height of the subject, and waist circumference of the subject.

The quantitative or qualitative parameter associated with the health state or condition of the at least one kidney of the subject may include a value associated with an estimated glomerular filtration rate of the subject, e.g., an estimated glomerular filtration rate score of the subject and an arithmetically-related score/value.

In some embodiments, step 104 includes: comparing the quantitative or qualitative parameter associated with the health state or condition of the at least one kidney of the subject with reference parameter data (reference parameter value(s) or range(s)) to determine whether the subject has a kidney disease. In some embodiments, step 104 further includes: classifying, based on the comparing, a stage or a severity of the kidney disease.

Preferably, in these embodiments, the kidney disease includes a chronic kidney disease.

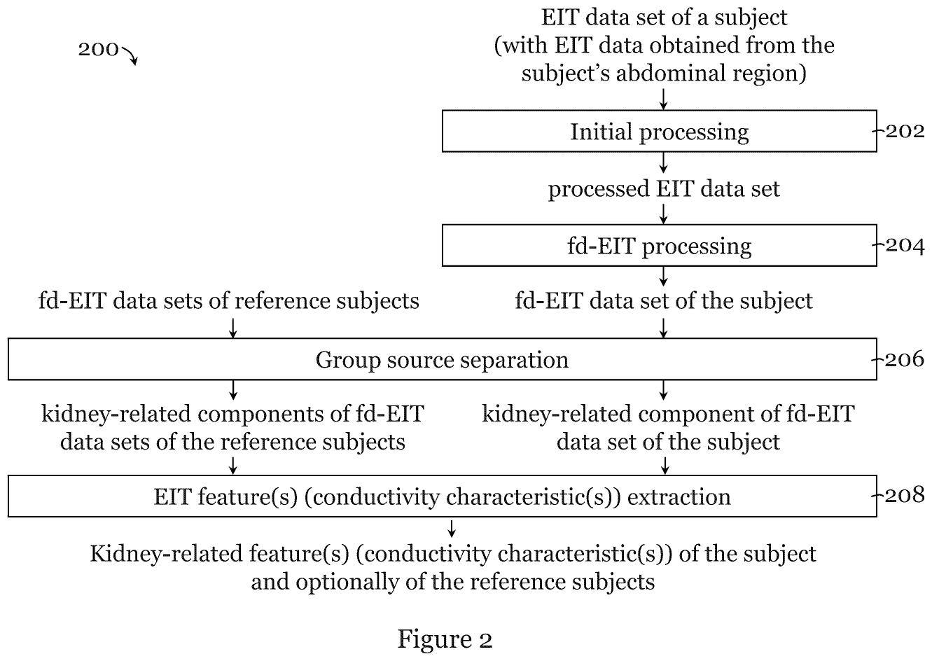

FIG. 2 shows a computer-implemented EIT data processing method 200 in some embodiments of the invention.

The method 200 includes, in step 202, performing an initial processing (pre-processing) on a EIT data set of a subject to obtain a processed EIT data set. The EIT data set includes EIT data obtained from the subject's abdominal region. The EIT data set may be obtained using the method 100 described above with reference to FIG. 1. The initial processing may include the processing mentioned with respect to step 102, (i), in the method 100 described above with reference to FIG. 1.

The method 200 includes, in step 204, performing a fd-EIT processing operation on the processed EIT data set to obtain a fd-EIT data set of the subject. The fd-EIT processing operation may include the processing mentioned with respect to step 102, (ii), in the method 100 described above with reference to FIG. 1. The fd-EIT processing operation may be other fd-EIT method known in the art to obtain conductivity maps.

The method 200 includes, in step 206, performing a group source separation operation using the fd-EIT data set of the subject and fd-EIT data sets of reference subjects (kidney-healthy subjects). The main aim of the group source separation operation is to separate or extract the source signals (i.e., the signals or data related to the kidney(s)) from the fd-EIT data set of the subject and fd-EIT data sets of reference subjects, which may include signals of other organs or tissues. The result of step 206 is the obtaining of kidney-related components of fd-EIT data sets of the reference subjects and kidney-related component of fd-EIT data set of the subject. In one example, if the one fd-EIT data set of the subject and two fd-EIT data sets of reference subjects, then after the group source separation operation, one set of data (a map) containing kidney-related component of fd-EIT data set of the subject and two sets of data (two maps) containing kidney-related components of fd-EIT data set of the reference subjects will be obtained. The group source separation operation may include the processing mentioned with respect to step 102, (iii), in the method 100 described above with reference to FIG. 1.

The method 200 includes, in step 206, performing EIT feature(s) extraction operation using the kidney-related components of fd-EIT data sets of the reference subjects and kidney-related component of fd-EIT data set of the subject. Here, the EIT feature(s) correspond to the conductivity characteristic(s) or feature(s). The result of step 206 is the obtaining of kidney-related feature(s) (conductivity characteristic(s)) of the subject and optionally of the reference subjects. The EIT feature(s) extraction operation may include the processing mentioned with respect to step 102, (iv), in the method 100 described above with reference to FIG. 1. In one example, if there exists one set of data (a map) containing kidney-related component of fd-EIT data set of the subject and two sets of data (two maps) containing kidney-related components of fd-EIT data set of two reference subjects, then one or more of these maps will be processed to determine the kidney-related feature(s). For example, a statistical operation may be performed on the entire map of containing kidney-related component of fd-EIT data set of the subject to obtain an average, mean, median, etc., of conductivity of the map. For example, a statistical operation may be performed on only part of the map of containing kidney-related component of fd-EIT data set of the subject to obtain an average, mean, median, etc., of conductivity of that part of the map. For example, a statistical operation may be performed on all of the maps containing kidney-related components of fd-EIT data set of the subject and the reference subjects to obtain an average, mean, median, etc., of conductivity of the aggregate of maps. For example, a delineation operation (e.g., thresholding) may be performed on the map of containing kidney-related component of fd-EIT data set of the subject to define a region of interest and obtain an average, mean, median, etc., of conductivity of that region of interest of the map. These can all be obtained as the EIT feature(s).

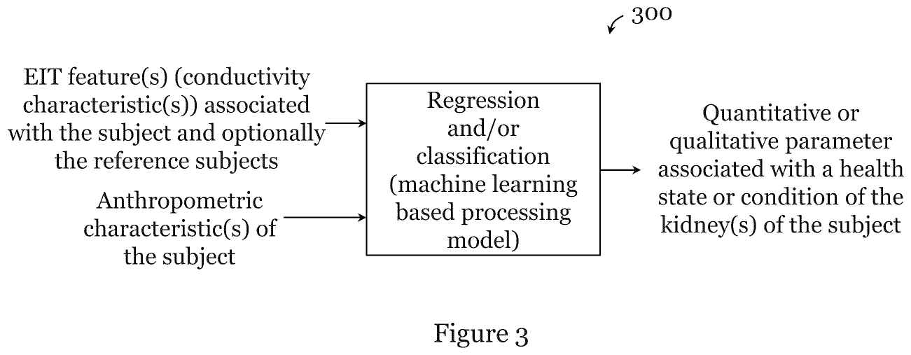

FIG. 3 shows a computer-implemented EIT data processing method 300 in some embodiments of the invention. The method 300 includes processing the EIT feature(s) (conductivity characteristic(s)) associated with the subject and optionally the reference subjects and the anthropometric characteristic(s) of the subject using a regression and/or classification model (e.g., machine learning based) to determine a quantitative or qualitative parameter associated with a health state or condition of the kidney(s) of the subject. The EIT feature(s) can be those in the methods 100, 200 described above with reference to FIGS. 1 and 2. The anthropometric characteristic(s) can be those in the method 100 described above with reference to FIG. 1.

The following provide some examples containing embodiments of the invention. These embodiments maybe considered as a more specific implementation of the method 100 in FIG. 1.

### Example 1

Example 1 can be considered as a specific example implementation of the method 100 in FIG. 1.

The example uses EIT to determine health condition of a kidney of a subject (e.g., humans, animals). Some embodiments of the invention classify CKD related conductivity characteristics.

In this example: the feasibility of ex-vivo conductivity measurement is tested on pig kidneys using an EIT device (e.g., a portable EIT device), the possibility of employing portable EIT on detecting renal function changes in-vivo on CKD patients is investigated, and conductivity characteristics at different CKD stages with frequency differencing EIT approach are also investigated.

### 1. Materials and Methods

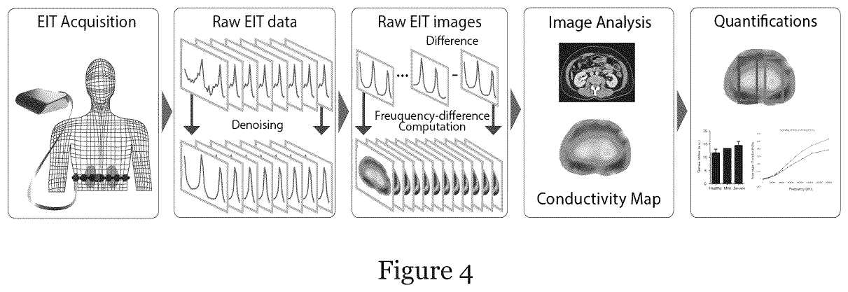

FIG. 4 shows an example operation including EIT data acquisition, processing, and analysis in this embodiment (for the in vivo experiments).

### 1.1 Subjects

10 healthy subjects and 88 clinical diagnosed CKD patients are tested in this example. Corresponding demographics and physical characteristics, including gender, age, weight, height, waist circumference, for the subjects are collected. In this example, the subjects have undergone both eGFR measurement and EIT-kidney assessment.

### 1.2 EIT Data Acquisition

All EIT examinations are performed with a portable EIT with five main modules: power management module to provide constant power supply to all other electronic modules through the power socket or the Li-ion battery, current generation module to generate AC of 1 mApp and a voltage amplitude of 1 Vpp, a signal distribution and readout module to introduce the generated current to the subject via the electrodes (e.g., 16-electrode belt) using a set of CMOS multiplexers, a data acquisition module is the analog front-end that acquires, measures, amplifies the differential voltage from the electrodes, and a control and output module consists of an analog-to-digital converter (ADC), an MCU for programing device outputs matching the desired measurement paradigm.

To test the feasibility to capture conductivity changes in vivo, ex-vivo EIT is performed on fresh pig kidney. EIT measurements are performed with saline water phantom (0.9% physiologic saline solution) with 16 silver electrodes. Current stimulation is induced at multiple frequencies ranging from 14 kHz to 200 kHz. EIT measurement is performed with empty water phantom with 33 frames per seconds (fps) to verify the functionality of the portable EIT system. Then, EIT measurements are repeated with fresh pig kidneys placed into water phantom

For in-vivo EIT measurements, the 98 subjects are tested. CKD stages are classified with the extracted eGFR scores from their blood serum samples [Stage 1 CKD (eGFR >90); Stage 2 CKD (eGFR: 61-90); Stage 3 CKD (eGFR: 31-60); Stage 4 CKD (eGFR: 15-30) and Stage 5 CKD (eGFR <15)]. EIT measurements are performed at 33 fps with two current injection frequencies (33.6 kHz and 100 kHz) using a portable EIT system and customized electrode belt consists of sixteen equally spaced gel-electrodes. The electrode belt is circumferentially positioned on the abdominal (upper abdominal) region. The contact of gel-electrodes is then manually checked before starting the EIT measurement, indicating by the low electrode-skin contact impedance. The subjects are asked to stay still and breathe normally throughout the EIT measurements.

### 1.3 Data Quantification & Analysis

In this embodiment, an alternating current is injected sequentially between all adjacent electrode pairs and the potential differences are measured across other 13 adjacent electrode pairs. A data frame consisting of 208 (16× 13) differential voltage measurements is acquired at a rate of 33 frames per second. Individual data frames are denoised by setting outlier voltage values above and below system thresholds to the corresponding value in the reference data frame. The EIT images are reconstructed from the denoised data frames and reference voltage data frame using one-step linear Gauss-Newton solver, with a regularization matrix based on the Newton's one-step error reconstructor prior with p=0.35 and a regularization hyperparameter of λ2=0.005.

Mean conductivity map is computed for each set of time-series images at each frequency. Frequency differencing is then performed by subtracting the conductivity maps at 33.6 kHz and 100 kHz. Kidney related conductivity values are then extracted from region of interest around kidney regions near bottom one-third of the reconstructed frequency differencing conductivity map. Conductivity changes between frequencies are computed and further regressed with individual biometrics. Predict eGFR values are further computed from the measured conductivity changes and compared with the standard eGFR scores to evaluate its robustness on classifying CKD stages.

### 2. Result

### 2.1 Ex-Vivo EIT Measurements on Pig Kidney with Water Phantom

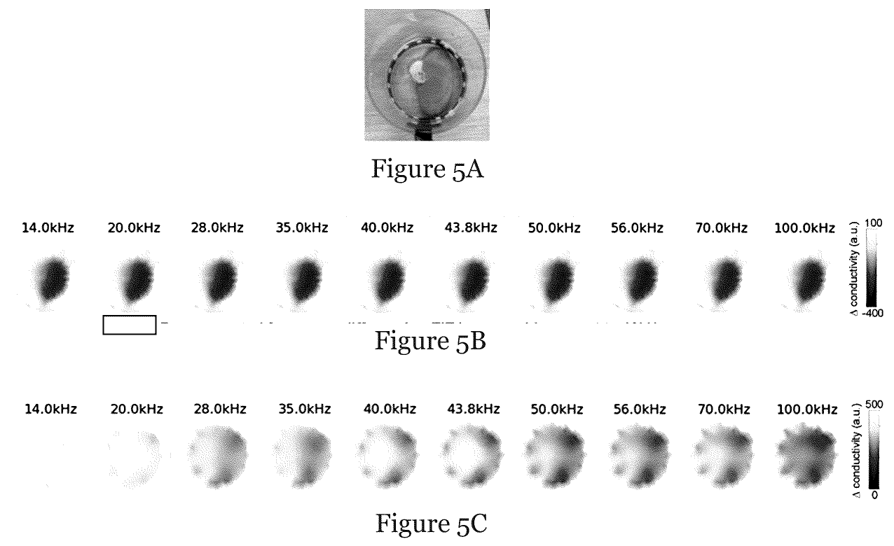

To test the feasibility conductivity measurements with EIT in vivo, an ex-vivo EIT is performed on fresh pig kidneys immersed in a water phantom. FIG. 5A shows a picture of a water phantom with the pig kidney.

FIG. 5B shows EIT images of pig kidney immersed in water phantom acquired by portable EIT device with respect to empty water phantom reference (multiple stimulation frequencies are applied for EIT measurements). FIG. 5C shows frequency difference EIT (fd-EIT) images of water phantom with pig kidney with empty 10 KHz EIT image as reference.

The reconstructed images with empty water phantom reference show the kidney contour (FIG. 5B), demonstrating the capability of portable EIT device to detect and localize the pig kidney inside physiologic saline medium. This demonstrates the sufficient penetrating power of portable EIT, with excited electric field at different frequencies range from 14 kHz to 200 kHz by a small ac current injection (1 mApp). Besides, frequency differencing conductivity maps are computed at multiple frequencies 10 KHz EIT images (FIG. 5C). The measured conductivity of the pig kidney is increased with increasing current simulation frequency, demonstrating the increased conductivity of kidney tissue over frequency change. Further, the fd-EIT images suggest the optimal stimulation frequencies for kidney imaging range from 28 kHz to 100 kHz. This result verifies the feasibility of applying fd-EIT in-vivo to measure regional conductivity characteristics on human subjects.

### 2.2 Conductivity Changes Cross Difference CKD Stages Predicts eGFR

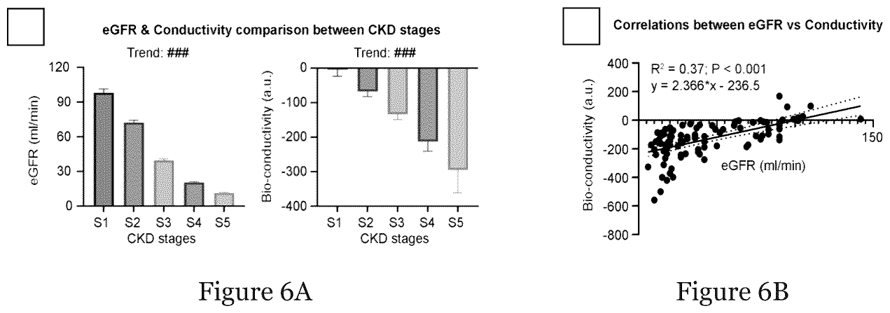

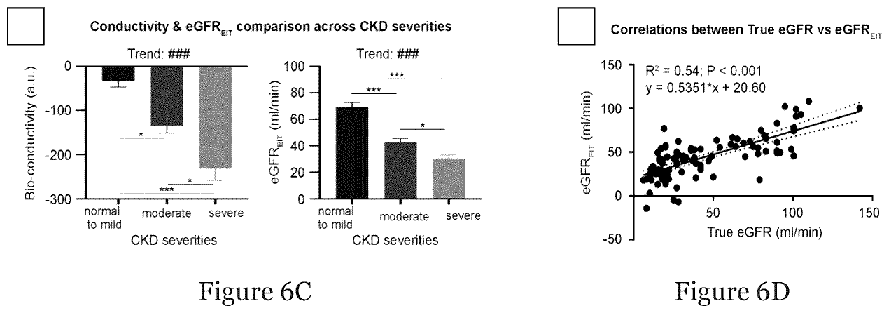

FIGS. 6A to 6D show that in this example the portable EIT device can discriminate CKD severities with EIT-predicted eGFR scores (eGFREIT) by conductivity measurements.

Specifically, FIG. 6A shows comparisons of standard eGFR values (left) and EIT-measured conductivity changes (right) between healthy subjects and patients across CKD stages [S1: stage 1 CKD (GFR: >90); S2: stage 2 CKD (GFR: 60-89); S3: stage 3 CKD (GFR: 30-59); S4: stage 4 CKD (GFR: 15-29) ; S5: stage 5 CKD (GFR: <15)]. Error bars indicate SEM of each group.

Specifically, FIG. 6B shows the correlations of eGFR values with EIT-measured conductivity changes. The eGFR values are significantly correlated to the portable EIT-kidney device measured conductivity changes (P<0.001).

Specifically, FIG. 6C shows comparisons of EIT measured conductivity changes (left) and EIT-predicted eGFR (right) across severities of CKD [i.e., Normal to Mild (eGFR: >60); Moderate (eGFR: 30-60); Severe (eGFR: <30)]. Statistical comparisons are performed using One-way-ANOVA by post-hoc Bonferroni post-hoc tests (*p<0.05, ** p<0.01, *** p<0.001) and trend analysis (###p<0.001). Error bars indicate±SEM.

Specifically, FIG. 6D shows the correlations of true eGFR with EIT-predicted eGFR across all subjects. The predicted eGFREIT scores are significantly associated with the true eGFR scores (R2=0.54; P<0.001).

EIT measurements are performed on 98 recruited patients with different stages of CKD (S1: n=16; S2: n=15; S3: n=20, S4: n=36 and S5: n=11) at two frequencies (33.6 kHz and 100 kHz) using a portable EIT device with an electrode belt consisting of 16 gel electrodes. The detected mean conductivity changes are −3.9, −67.5, −133.5, −212.6, and −294.7 (a.u.) in patients with CKD Stage 1, 2, 3, 4 and 5, respectively (FIG. 6A). The trend analysis shows that patients with more advanced stages of CKD show significant reduction in bio-conductivity. The observation is further confirmed by the positive correlation between true eGFR with reduction in conductivity (R²=0.37, p<0.001) (FIG. 6B). In addition, statistical comparisons and trend analysis are performed on the conductivity characteristics between severities of CKD (i.e., normal to mild, moderate, and severe). The detected conductivity reduction by fd-EIT showed significant differences between severity levels (FIG. 6C). Furthermore, EIT-predicted eGFR (eGFREIT) is computed by the captured conductivity changes with individual biometrics (FIG. 6D). The eGFREIT shows significant correlation with eGFR measured by standard blood tests (R²=0.54, p<0.001), indicating the fd-EIT measured conductivity characteristics are useful for predicting standard eGFR value. The results in this example highlight the potential of portable EIT device on predicting the eGFR scores and potentially beneficial to early screening or chronic treatment monitoring of CKD.

### 3. Discussion

This example investigates a non-invasive approach for CKD evaluation by examining the conductivity characteristics. It is found that subjects with later CKD stages show lower eGFR and significant greater conductivity decrease. This may be due to reduced renal blood perfusion, restricted fluid diffusion, presence of fibrotic tubules and tubular atrophy during kidney disease progression. These renal functional deterioration and kidney structural changes could lead to the observed conductivity decreases.

This example investigates the conductivity characteristics of different CKD stages with portable frequency differencing EIT device. A significant correlation between EIT-predicted eGFR by captured conductivity changes and standard eGFR is found. Such renal function assessments with portable EIT device demonstrates the potential to ameliorate the detection and classification of CKD into a portable, accessible, self-administrable home-based process.

### Example 2

Example 2 can be considered as a specific example implementation of the methods 100-300 in FIGS. 1-3.

In this example: a data processing pipeline to extract the kidney signals from in vivo EIT data is provided, and a regression model to estimate the eGFR of CKD patients using EIT features and the age only is provided. The regression result is used to classify the CKD stage of the patient.

### 1. Materials and Methods

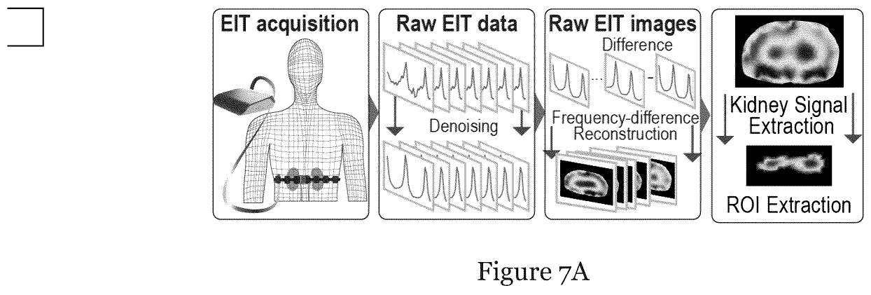

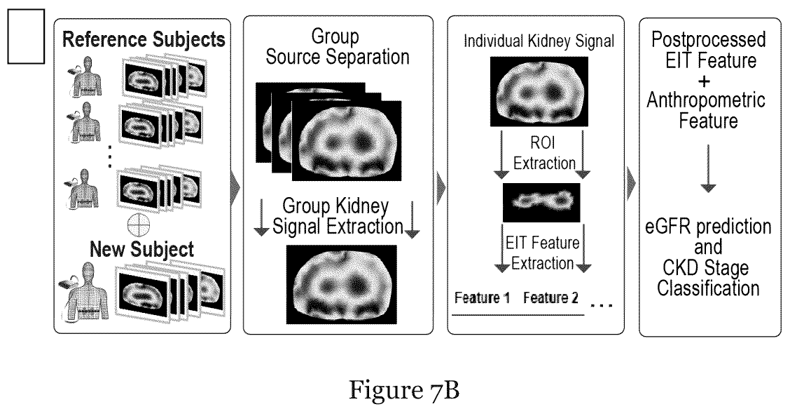

FIGS. 7A and 7B show an example operation including EIT data acquisition, processing, and analysis in this embodiment. Specifically, FIG. 7A shows an example of EIT data acquisition, data preprocessing and data analysis pipeline; FIG. 7B shows detailed illustration of eGFR prediction pipeline including the group source separation.

### 1.1 Subject

21 healthy individuals (controls) and 54 clinical diagnosed CKD patients are tested in this example. Corresponding demographics and physical characteristics, including gender, age, weight, height, waist circumference, for the subjects are collected. In this example, the subjects have undergone both eGFR measurement and EIT-kidney assessment.

### 1.2 EIT Acquisition

All EIT experiments in this example are conducted using a PVC electrode belt connected to a palm sized portable EIT console through an HDMI cable. The EIT console is connected to a computer with specialized software to collect, visualize and save the collected raw EIT data and system information for further data processing and analysis. The electrode belt consists of 16 modular electrode holders, each containing a printed circuit board that can be connected to 1 gel electrode. The electrode belt is placed circumferentially on the abdominal (upper abdominal) region. Throughout the whole EIT data acquisition process, the subjects are asked to breathe normally and to stay still.

In total, there are 208 (16 injection pairs×13 measurement pairs per injection pair) stimulation-measurement at each frequency. Each measurement is measured at a frame rate of 33 fps. 24 frequencies, in the range of 28 kHz to 300 kHz, are used in the EIT measurement and analysis process.

To acquire ground truth eGFRs, blood serum and urine samples are collected from each subject. Creatinine level, evaluated using the blood serum samples, is then used to derive the eGFR score of the subject with the known equation:

$$eGFR = 141 \times min {( {S}_{Cr} / κ , 1 )}^{α} \times max ( {S}_{Cr} / κ , 1 ) - 1.209 \times {0.993}^{Age} \times {1.018}_{[ if female ]} \times {1.159}_{[ if black ]}$$

where eGFR is the estimated glomerular filtration rate (mL/min/1.73 m²), S_{Cr} is the standardized serum creatinine (mg/dL), κ=0.7 for females or κ=0.9 for males, α=−0.329 for females or α=−0.411 for males, min indicates the minimum of S_{Cr}/K or 1, max indicates the maximum of S_{Cr}/K or 1, Age represents age of subject in years.

CKD stages are classified according to the value of eGFRs extracted from blood serum samples with the following criteria: Stage 1 CKD: (eGFR >90); Stage 2 CKD: (eGFR: 60-89); Stage 3 CKD: (eGFR: 30-60); Stage 4 CKD: (eGFR: 15-30); Stage 5 CKD: (eGFR: <15). The CKD stages are also grouped in terms of severity according to the following scheme: normal to mild (Stage 1-2), moderate (Stage 3) severe (Stage 4-5).

### 1.3 Data Pre-processing

EIT reconstruction is conducted in Python using the library pyEIT and customized functions. Frequency difference EIT is used in this example, with reference frequency at 30 kHz and the other 23 frequencies ranging from 28 kHz to 300 kHz used for frequency difference reconstruction.

To ensure the quality of data for the purpose of further analysis, a measurement quality classifier and a reconstruction algorithm for arbitrary stimulation-measurement patterns are developed. For each frame, the classifier filters data to remove undesired effects due to uncontrollable events such as faulty measurements due to subject movements.

For each subject and each frequency, the mean of all frames is taken, all electrodes that are corrupted in any one of the frames are labelled and the corresponding stimulation-measurements are removed after taking the mean across frames. Since a reconstruction algorithm for arbitrary stimulation-measurement patterns is developed, we are able to reconstruct the conductivity images after the electrodes involved in faulty measurements are removed.

### 1.4 Data Analysis

Group source separation (FIG. 7B) is used to isolate signals from different internal tissues out of the reconstructed images of each individual. Frequency differences conductivity images at all contrast frequencies are used for the group source separation. Healthy subjects' data are used as reference group to assist in isolating the signals because the electrical responses from healthy subjects are more consistent in comparison to including all subjects from a variety of CKD stages, including subjects who experienced nephrotomy.

After isolating the signals sources, the source of the kidney signal is determined from the group separation result. From this group result, the individual kidney source is extracted. After the group source separation, the signal from the kidneys is the strongest amongst all other signals in the extracted kidney image component. The region of interest (ROI), i.e., the kidneys, is then extracted from the individual source.

After the kidney signal and the ROIs are extracted, EIT related features are generated, including but not limited to the mean conductivity within, outside the ROIs and the ratio between them. The data are split into train set and test set with 60 and 15 data points respectively in a stratified manner. A linear regression model is trained with the train set is evaluated using 5-fold cross validation.

Statistical comparisons are performed on CKD stages and severity classification using one-way ANOVA followed by multiple comparisons Bonferroni post-hoc tests (*p<0.05, ** p<0.01, *** p<0.001).

The principle and operation of the group source separation has been described with reference to FIG. 2 hence is not repeated here.

### 2. Result

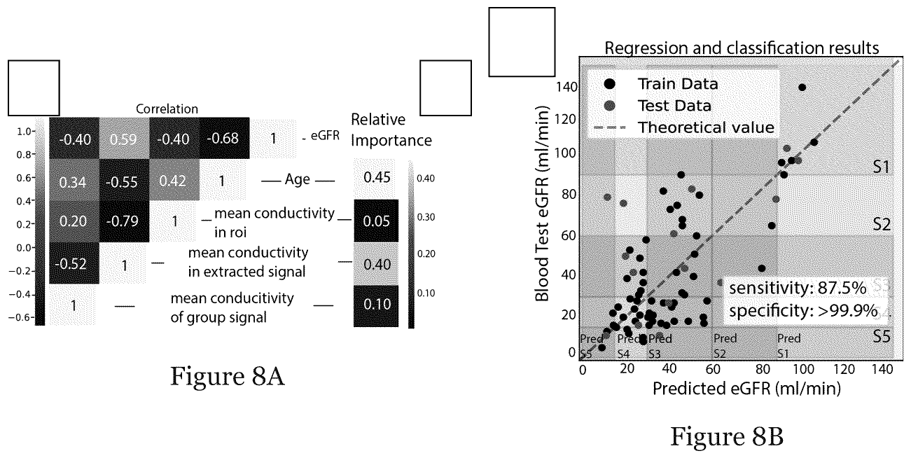

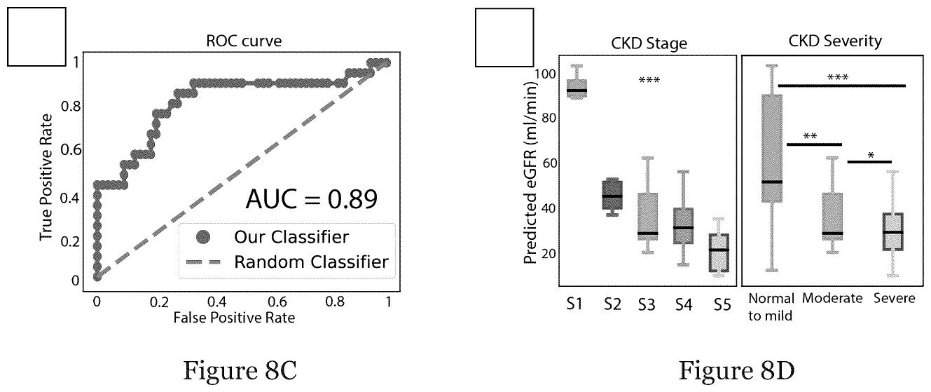

FIG. 8A shows the linear correlation coefficients and relative importance of EIT-features in the prediction of eGFR. FIG. 8B shows the correlation and classification specificity and sensitivity of the eGFR regression model. FIG. 8C shows a ROC curve of the regression model and the classification scheme. FIG. 8D shows comparison of EIT-predicted eGFR across CKD stages and CKD severity.

### 2.1 Correlation

It is found that the eGFR is correlated with mean conductivity of the group kidney source, the individual kidney source, and the kidney ROI with linear correlation coefficients of −0.4, 0.59 and −0.4. In addition to the EIT features, it is also found that the age is linear correlated to the eGFR with a coefficient of −0.68. It is found that the mean conductivity within the kidney ROIs is negatively linearly correlated to the mean conductivity of the individual kidney signal image with coefficient of −0.79. Please see FIG. 8A for details. Note that in FIG. 8A, the “mean conductivity in roi” refers to the mean of the ROI only in the conductivity map of the subject after group source separation (i.e., the lower image in the third box in FIG. 7B), the “mean conductivity in extracted signal” refers to the mean of the entire conductivity map of the subject after group source separation, and the “mean conductivity of group signal” refers to the mean of all conductivity maps of the subject and reference subjects after group source separation.

### 2.2 Regression Model

In this example the EIT features are fitted together with the age using a Lasso algorithm. The age has the highest relative importance of 0.45 among all the features, while the mean conductivity in the individual kidney source, in the group source, and in the ROI are 0.40, 0.10 and 0.05, respectively (FIG. 8A). The model has a cross-validated R² score of 0.4, which means that the EIT-feature prediction model has a significant correlation with the eGFR derived from blood serum and urine test.

### 2.3 CKD Classification

The CKD stages are obtained from the eGFR predicted from the regression model by the following criteria: Stage 1 CKD: (eGFR >90); Stage 2 CKD: (eGFR: 60-89); Stage 3 CKD: (eGFR: 30-60) ; Stage 4 CKD: (eGFR: 15-30); Stage 5 CKD: (eGFR: <15). Considering Stage 1 CKD as healthy and Stages 3, 4 and 5 CKD as non-healthy, a specificity of >99.9% and sensitivity of 87.5% in obtained (FIG. 8B). The area under curve (AUC) of the receiver operating characteristic (ROC) curve is found by setting the ground truth threshold eGFR for being healthy at 60 ml/min while varying the classification threshold for the classification from predicted eGFR. An AUC of 0.89 is obtained (FIG. 8C).

### 3. Discussion

This example demonstrates an eGFR estimation model and a CKD stage classification scheme using a portable, self-administrative EIT device. The operation of this device does not require dedicated professionally trained staff and clinical environment. Furthermore, this imaging device is non-invasive, ionizing-radiation-free and is cost-effective. This device can be used for medical screenings, for early chronic kidney disease diagnosis and longitudinal renal function monitoring without the need of public health services. Therefore, the device can enhance the quality and extend the area of application of telemedicine to renal function monitoring and chronic kidney diseases. Further, due to the portability and user-friendliness, the device can provide community-based CKD screening for individuals in locations that could be missed by public healthcare system.

In this example it is found that the mean conductivity in the individual extracted signal has a very negative linear correlation with the mean conductivity in ROI while having a relative importance of 0.4 in the Lasso model and a linear correlation coefficient of 0.59 with the eGFR. This suggests that the mean conductivity in the individual extracted signal is dominated by signals related to kidney functions.

The evaluation of classification specificity and sensitivity is based on 21 healthy subjects. A simulation based on the prediction error and a population eGFR distribution of the existing Lasso model is generated. Data from NHANES are used as the population eGFR distribution. Corresponding simulation reul

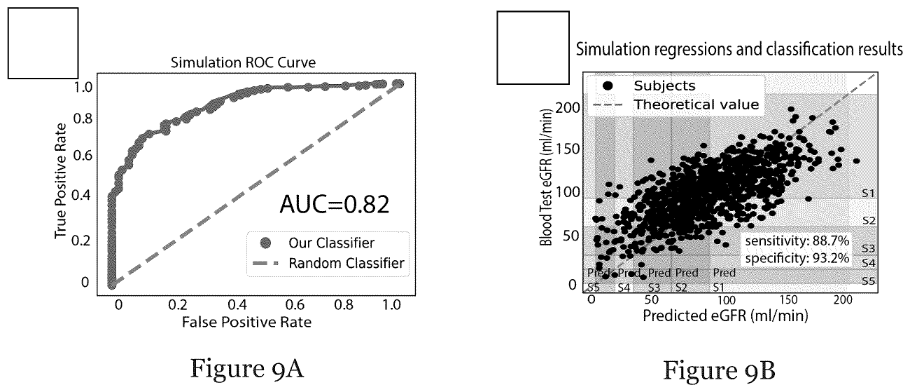

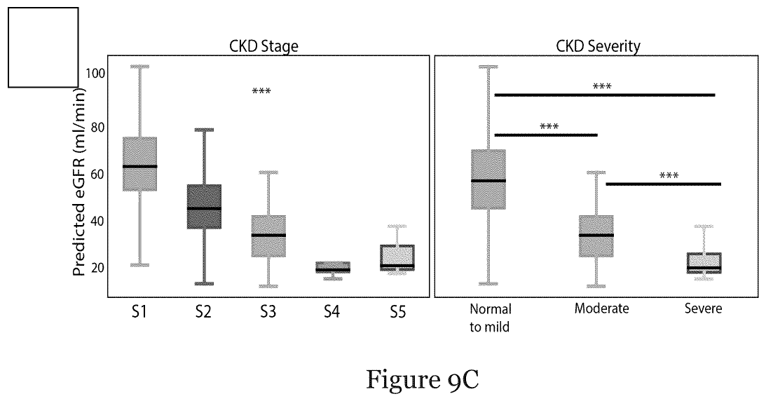

FIGS. 9A to 9C show the simulation results based on existing population eGFR distribution and the error of the proposed model in this example. Specifically, FIG. 9A shows an ROC curve of the simulation, FIG. 9B shows the correlation and classification specificity and sensitivity of simulation, FIG. 9C shows the comparison of the simulated EIT-based eGFR estimation across CKD stages and CKD severity.

An ROC curve with AUC=0.82 is obtained (FIG. 9A). The resulting sensitivity and specificity are 88.67% and 93.24% using N=1000 simulated data points (FIG. 9B).

In summary, in this example, clinical data on 54 CKD patients and 21 healthy subjects with a portable EIT device. This example provides a data processing pipeline with a group source separation algorithm that isolates the kidney signals from raw EIT data. There is found significant correlations between standard eGFR and eGFR predicted from a linear model using EIT features and the age. CKD stages are classified from the estimated eGFR using the proposed model and 87.5% sensitivity and >99.9% specificity are obtained. This renal function assessment example demonstrates the feasibility of EIT to be used in the field of telemedicine as a non-invasive approach for early CKD diagnosis and potential for longitudinal CKD monitoring.

### Example Systems

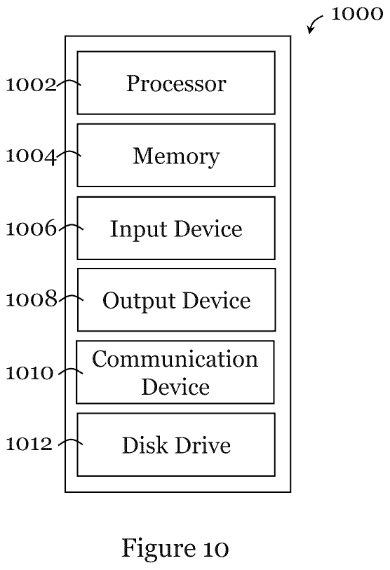

FIG. 10 shows an example data processing system 1000 that can be used to perform one or more of the method embodiments (partly or entirely) in some embodiments of the invention. The data processing system 1000 may be included in a server, a mobile device, a computer, etc. The data processing system 1000 generally includes suitable components necessary to receive, store, and execute appropriate computer instructions, commands, and/or codes. The main components of the data processing system 1000 are a processor 1002 and a memory (storage) 1004. The processor 1002 may include one or more: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries configured to interpret and/or to execute program instructions and/or to process signals and/or information and/or data. The processor 1002 can be used to perform machine learning based processing and non-machine learning based processing. The memory 1004 may include one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations. Appropriate computer instructions, commands, codes, information and/or data (e.g., any one or more of: EIT data, processed EIT data, fd-EIT data, kidney-related components of fd-EIT data, one or more machine learning based models, anthropometric characteristic(s) of subjects, EIT feature(s) associated with the subjects, quantitative or qualitative parameters associated with a health state or condition of the kidney(s) of the subjects) may be stored in the memory 1004. Computer instructions for executing or facilitating executing the method embodiments of the invention may be stored in the memory 1004. The processor 1002 and memory (storage) 1004 may be integrated or separated (and operably connected). Optionally, the data processing system 1000 further includes one or more input devices 1006. Examples of such input device 1006 include: keyboard, mouse, stylus, image scanner, microphone, tactile/touch input device (e.g., touch sensitive screen), image/video input device (e.g., camera), etc. Optionally, the data processing system 1000 further includes one or more output devices 1008. Examples of such output device 1008 include: display (e.g., monitor, screen, projector, etc.), speaker, headphone, earphone, printer, additive manufacturing machine (e.g., 3D printer), etc. The display may include a LCD display, a LED/OLED display, or other suitable display, which may or may not be touch sensitive. The data processing system 1000 may further include one or more disk drives 1012 which may include one or more of: solid state drive, hard disk drive, optical drive, flash drive, magnetic tape drive, etc. A suitable operating system may be installed in the data processing system 1000, e.g., on the disk drive 1012 or in the memory 1004. The memory 1004 and the disk drive 1012 may be operated by the processor 1002. Optionally, the data processing system 1000 also includes a communication device 1010 for establishing one or more communication links (not shown) with one or more other computing devices, such as servers, personal computers, terminals, tablets, phones, watches, IoT devices, or other wireless computing devices. The communication device 1010 may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection, or other wired or wireless communication interfaces. Transceiver may be implemented by one or more devices (integrated transmitter(s) and receiver(s), separate transmitter(s) and receiver(s), etc.). The communication link(s) may be wired or wireless for communicating commands, instructions, information and/or data. In one example, the processor 1002, the memory 1004 (optionally the input device(s) 1006, the output device(s) 1008, the communication device(s) 1010 and the disk drive(s) 1012, if present) are connected with each other, directly or indirectly, through a bus, a Peripheral Component Interconnect (PCI), such as PCI Express, a Universal Serial Bus (USB), an optical bus, or other like bus structure. In one embodiment, at least some of these components may be connected wirelessly, e.g., through a network, such as the Internet or a cloud computing network. A person skilled in the art would appreciate that the data processing system 1000 shown in FIG. 10 is merely an example and that the data processing system 1000 can in other embodiments have different configurations (e.g., include additional components, has fewer components, etc.).

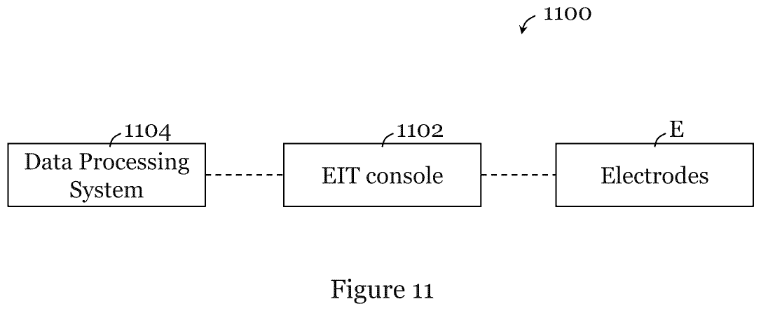

FIG. 11 shows an EIT system 1100 in some embodiments of the invention. The EIT system 1100 generally includes an EIT console 1102 (which may or may not be portable), electrodes E electrically connected with the EIT console 1102 to obtain signals from the subject, and a data processing system 1104 operably connected with the EIT console 1102. In one example, the EIT console 1102 is mainly used to control signal transmission and receive, to and from the body of the subject, via the electrodes E. In one example, the data processing system 1104 is mainly used to process the signals obtained by the EIT console 1102 via the electrodes E. In one example, the data processing system 1104 may be at least partly integrated with the EIT console such that the data processing can be at least partly performed at the EIT console. In one example, the data processing system 1104 may be the system 1000 of FIG. 10.

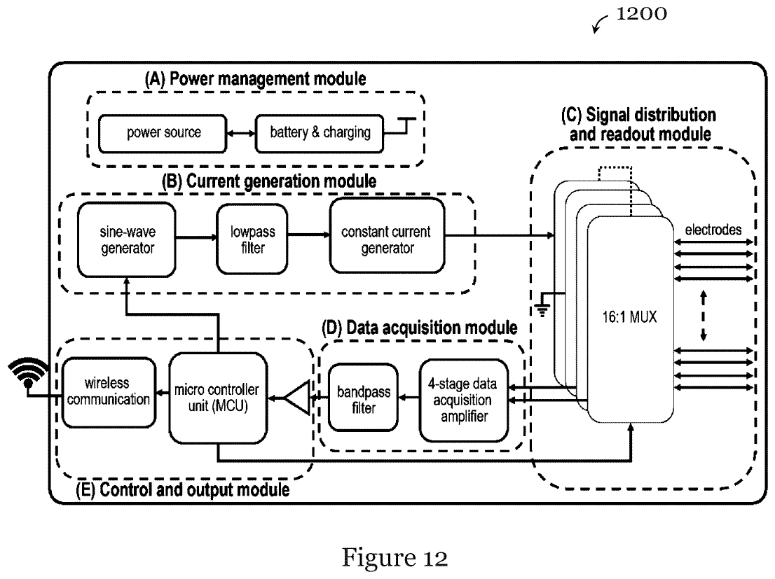

FIG. 12 shows example EIT console used for EIT data acquisition in some embodiments of the invention. In this example the EIT console is portable. Referring to FIG. 12, the EIT console generally includes a power management module for constant power supply, a current generation module for alternating current generation, a signal distribution and readout module for current injection and voltage readout, a data acquisition module for potential difference measurement, amplification and acquisition, and a control and output module for module coordination, data processing and cloud-server communication. As illustrated in FIG. 12, the EIT console includes an EIT console having 5 major modules:
Power management module that provides power supply to all other modules through the power socket or battery.
Current generation module that primarily includes a sine wave generator and a constant current generator successively to generate an alternating current of 1 mApp and a voltage amplitude of 1 Vpp. A low-pass filter may be included to suppress total harmonic distortion and ambient electromagnetic interference (e.g., power line noise).
Signal distribution and readout module that introduces the generated current to the subject via the 16-electrode belt using a set of CMOS multiplexers (MUXs). Four MUXs are used, in which two MUXs are employed for current injection and the other two for voltage readout. The MUXs are configured into the adjacent-scan pattern through the microcontroller unit (MCU).
Data acquisition module that is the analog front-end (AFE) that acquires, measures and amplifies the potential differences from the electrodes. The AFE comprises a four-stage wide input differential amplifier with high common-mode rejection ratio (CMRR), and a bandpass filter.
Control and output module that includes an analog-to-digital converter (ADC), a MCU and a wireless communication chip. The potential differences obtained from the data acquisition module are digitized by the ADC, processed in the MCU unit, and transferred to an external device (server, phone, computer, etc.) for image reconstruction and processing.

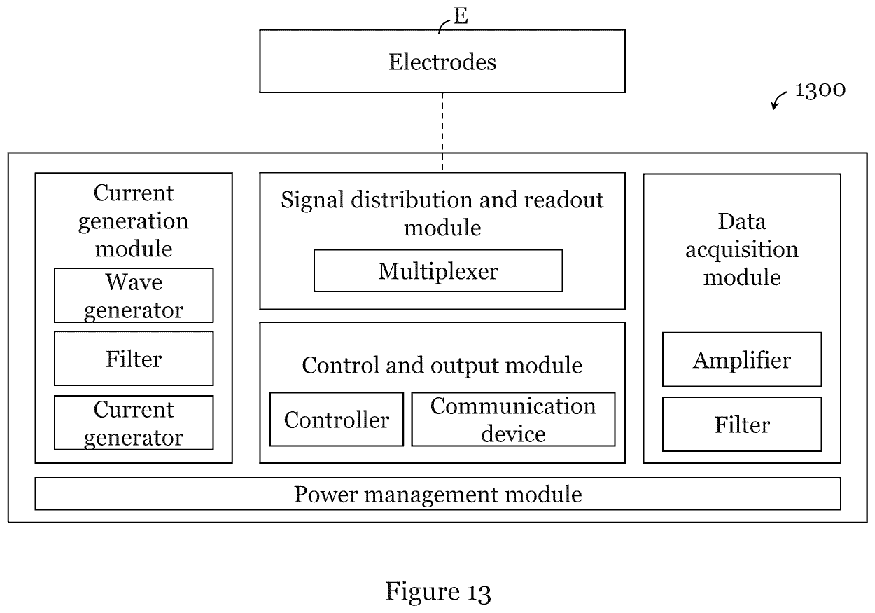

FIG. 13 is a block diagram of another example EIT console 1300 in some embodiments of the invention. The EIT console 1300 is basically a generalized version of the console 1200. The EIT console 1300 may or may not be portable.

Although not required, the embodiments described with reference to the Figures can be implemented as an application programming interface (API) or as a series of libraries for use by a developer or can be included within another software application, such as a terminal or computer operating system or a portable computing device operating system. Generally, as program modules include routines, programs, objects, components and data files assisting in the performance of particular functions, the skilled person will understand that the functionality of the software application may be distributed across a number of routines, objects and/or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include (but not limited to) any appropriate arrangement of computer or information processing hardware capable of implementing the function described.

It will be appreciated by a person skilled in the art that variations and/or modifications may be made to the described and/or illustrated embodiments of the invention to provide other embodiments of the invention. The described/or illustrated embodiments of the invention should therefore be considered in all respects as illustrative, not restrictive. Example optional features of the invention are provided in the summary and the description. Some embodiments of the invention may include one or more of these optional features (some of which are not specifically illustrated in the drawings). Some embodiments of the invention may lack one or more of these optional features (some of which are not specifically illustrated in the drawings). For example, the method embodiments of the invention are not limited for use in humans but can be use in other animals. The method can be applied for determining health state or condition of the kidney (one or both) of the subject can be extended and applied for determining health state or condition of other tissues and/or organs of any animal or human subjects. The EIT data acquisition need not be performed using a portable EIT device such as the ones illustrated—the EIT data can be obtained using other EIT devices. The data processing methods of the invention can be implemented on any suitable device or devices (including one or more of server, computer, phone, the EIT console (portable or not), etc.).

1. A computer-implemented method, comprising:
processing a EIT data set of a subject to determine one or more kidney-related conductivity characteristics of the subject; and
determining, based on at least the one or more determined kidney-related conductivity characteristics, a health state or condition of the at least one kidney of the subject.

2. The computer-implemented method of claim 1, wherein the determining comprises:
determining, based on at least the one or more determined kidney-related conductivity characteristics, whether the subject has a kidney disease, and optionally: further classifying a stage or a severity of the kidney disease.

3. The computer-implemented method of claim 2, wherein the determining comprises:
determining, based on at least the one or more determined kidney-related conductivity characteristics, a value associated with an estimated glomerular filtration rate of the subject, e.g., the estimated glomerular filtration rate of the subject.

4. The computer-implemented method of claim 1, wherein the determining comprises:
processing, at least, the one or more determined kidney-related conductivity characteristics of the subject and one or more anthropometric characteristics of the subject, using a machine learning based processing model, to determine a quantitative or qualitative parameter associated with the health state or condition of the at least one kidney of the subject.

5. The computer-implemented method of claim 1, wherein the determining comprises:
processing, using a machine learning based processing model,
(i) the one or more determined kidney-related conductivity characteristics of the subject,
(ii) one or more anthropometric characteristics of the subject, and
(iii) one or more determined kidney-related conductivity characteristics of one or more reference subjects and/or one or more determined kidney-related conductivity characteristics of a group containing the subject and the one or more reference subjects,
to determine a quantitative or qualitative parameter associated with the health state or condition of the at least one kidney of the subject.

6. The computer-implemented method of claim 4, wherein the machine learning based processing model comprises a regression model.

7. The computer-implemented method of claim 6, wherein the regression model comprises a linear regression model, such as a Lasso model.

8. The computer-implemented method of claim 7, wherein the one or more anthropometric characteristics comprise, or are related to, one or more of: age of the subject, weight of the subject, height of the subject, and waist circumference of the subject.

9. The computer-implemented method of claim 8, wherein the quantitative or qualitative parameter associated with the health state or condition of the at least one kidney of the subject comprises: a value associated with an estimated GFR of the subject, e.g., an estimated GFR score of the subject.

10. The computer-implemented method of claim 9, wherein the determining further comprises:
comparing the quantitative or qualitative parameter associated with the health state or condition of the at least one kidney of the subject with reference parameter data to determine whether the subject has a kidney disease.

11. The computer-implemented method of claim 10, wherein the determining further comprises:
classifying, based on the comparing, a stage or a severity of the kidney disease.

12. The computer-implemented method of claim 11, wherein the kidney disease is a chronic kidney disease.

13. The computer-implemented method of claim 1, wherein the EIT data set contains EIT data obtain from an abdominal region of the subject;
wherein the EIT data set is obtained by
(a) providing excitation signals at a frequency to the subject via electrodes attached to the abdominal region of the subject,
(b) measuring responsive signals received via the electrodes as a result of the providing of the excitation signals, and
(c) repeating steps (a) and (b) for a plurality of frequencies; and
wherein the EIT data set comprises a plurality of EIT data subsets each associated with a respective one of the plurality of frequencies.

14. The computer-implemented method of claim 13, wherein the processing comprises:
processing the EIT data set to obtain a processed EIT data set with a plurality of processed EIT data subsets;
processing the processed EIT data set to obtain a frequency difference EIT data set, the frequency difference EIT data set includes a plurality of frequency difference EIT data subsets;
performing a group source separation operation using the frequency difference EIT data set and one or more reference frequency difference EIT data sets of corresponding one or more reference subjects to determine kidney-related component of the frequency difference EIT data set and kidney-related component of each of the one or more reference frequency difference EIT data sets; and
performing a conductivity characteristics extraction operation using the kidney-related component of the frequency difference EIT data set and optionally the kidney-related component of each of the one or more reference frequency difference EIT data sets to determine at least the one or more kidney-related conductivity characteristics of the subject.

15. The computer-implemented method of claim 14, wherein the processing of the EIT data set comprises:
filtering and/or smoothing each of the plurality of EIT data subsets.

16. The computer-implemented method of claim 15, wherein the processing of the EIT data set comprises:
processing the EIT data set using a classifier model to determine respective performance of each of the plurality of electrodes, the performance being associated with quality of responsive signals or data obtained from the respective electrode; and
preventing the responsive signals or data obtained via any one or more of the plurality of electrodes determined to have insufficient performance from being included in the processed EIT data set.

17. The computer-implemented method of claim 16, wherein the processing of the processed EIT data set comprises:
determining, for each respective one or more of the plurality of processed EIT data subsets, respective difference between the respective processed EIT data subset and a reference EIT data subset, so as to obtain the plurality of frequency difference EIT data subsets each associated with a respective one of a difference between the respective processed EIT data subset and a reference EIT data subset.

18. The computer-implemented method of claim 17, wherein the reference EIT data subset comprises at least one of the plurality of processed EIT data subsets.

19. The computer-implemented method of claim 13, wherein the processing comprises:
processing the EIT data set to obtain a frequency difference EIT data set, the frequency difference EIT data set includes a plurality of frequency difference EIT data subsets;
performing a group source separation operation using the frequency difference EIT data set and one or more reference frequency difference EIT data sets of corresponding one or more reference subjects to determine kidney-related component of the frequency difference EIT data set and kidney-related component of each of the one or more reference frequency difference EIT data sets; and
performing a conductivity characteristics extraction operation using the kidney-related component of the frequency difference EIT data set and optionally the kidney-related component of each of the one or more reference frequency difference EIT data sets to determine at least the one or more kidney-related conductivity characteristics of the subject.

20. The computer-implemented method of claim 19, wherein the performing of the group source separation operation comprises:
performing a dimensionality reduction operation on the frequency difference EIT data set and one or more reference frequency difference EIT data sets of corresponding one or more reference subjects to determine kidney-related component of the frequency difference EIT data set and respective kidney-related component of each of the one or more reference frequency difference EIT data sets.

21.-29. (canceled)
